(19) United States
(12) Patent Application Publication
(10) Pub. No.: US 2010/0160747 A1
(43) Pub. Date: Jun. 24, 2010

(54) SELECTION OF PREFERRED SAMPLING LOCATION ON HAND VIA MINIMIZATION OF SAMPLING ERROR, AND OPTICAL ALIGNMENT FOR REPEATABLY SAMPLING TISSUE

(76) Inventors: Mark Ries Robinson, Albuquerque, NM (US); Andrea Magee, Albuquerque, NM (US); Michael J. Haass, Albuquerque, NM (US); Russell E. Abbink, Sandia Park, NM (US); William Radigan, Albuquerque, NM (US); V. Gerald Grafe, Corrales, NM (US)

Correspondence Address: The Grafe Law Office, P.C., P.O. Box 2689, Corrales, NM, 87048, US

(21) Appl. No.: 12/636,771

(22) Filed: Dec. 13, 2009

Related U.S. Application Data
(60) Provisional application No. 61/122,158, filed on Dec. 12, 2008.
(60) Provisional application No. 61/122,124, filed on Dec. 12, 2008.

(51) Int. Cl. A61B 5/1468; A61B 5/00

(52) U.S. Cl. 600/306; 600/316

(57) ABSTRACT

The present invention relates to measurements of material properties by determination of the response of a sample to incident radiation, and more specifically to the measurement of analytes such as glucose or alcohol in human tissue. The invention is particularly useful in connection with noncontact optical sampling of skin. Some example embodiments of the invention provide for selection of preferred sampling locations responsive to optically-determined characteristics of the tissue. Some example embodiments of the invention provide for precise and repeatable alignment of the tissue based on optically-determined characteristics of the tissue.

### CROSS-REFERENCE TO RELATED APPLICATIONS

This application claims priority to U.S. provisional application 61/122,158, filed Dec. 12, 2008; and to U.S. provisional application 61/122,124, filed Dec. 12, 2008; each of which is incorporated herein by reference.

### BACKGROUND OF THE INVENTION

This invention relates to measurements of material properties by determination of the response of a sample to incident radiation, and more specifically to the measurement of analytes such as glucose or alcohol in human tissue. The invention is particularly useful in connection with noncontact optical sampling of skin.

Noninvasive analyte measurement, especially noninvasive glucose measurement, has been a long-standing objective for many development groups. Several of these groups have sought to use near infrared spectroscopy as the measurement modality. To date, none of these groups has demonstrated a system that generates noninvasive glucose measurements adequate to satisfy both the U.S. Food and Drug Administration (“FDA”) and the physician community. Spectroscopic noise introduced by the tissue media is a principal reason for these failures. Tissue noise can include any source of spectroscopic variation that interferes with or hampers accuracy of the analyte measurement. Changes in the optical properties of tissue can contribute to tissue noise. The measurement system itself can also introduce tissue noise, for example changes in the system can make the properties of the tissue appear different. Tissue noise has been well recognized in the published literature, and is variously described as physiological variation, changes in scattering, changes in refractive index, changes in pathlength, changes in water displacement, temperature changes, collagen changes, and changes in the layer nature of tissue. See, e.g., Khalil, Omar: Noninvasive glucose measurement technologies: an update from 1999 to the dawn of the new millennium. Diabetes Technology & Therapeutics, Volume 6, number 5, 2004. Variations in the optical properties of tissue can limit the applicability of conventional spectroscopy to noninvasive measurement. Conventional absorption spectroscopy relies on the Beer-Lambert-Bouger relation between absorption, concentration, pathlength, and molar absorptivity. For the single wavelength, single component case:

I_{λ}=I_{λ,o}10^{-ε}^{_{λ}}^{lc}

a_{λ}=ε_{λ}lc

Where I_{λ,o} and I_{λ} are the incident and excident flux, is the molar absorptivity, c is the concentration of the species, and l is the pathlength through the medium. a is the absorption at wavelength (−log₁₀(I_{λ}/I_{λ,o}). These equations assume that photons either pass through the medium with pathlength l, or are absorbed by the molecular occupants.

Unfortunately, optical measurement of tissue does not match the assumptions required by Beer's law. Variations in tissue between individuals, variations in tissue between different locations or different times with the same individual, surface contaminants and varying surface topology and condition, interaction of the measurement system with the tissue, and many other real-world effects can prevent accurate optical measurements. There is a need for improvements in optical measurement methods and apparatuses that allow accurate measurements in real-world settings.

Noninvasive glucose measurement devices that sample forearm tissue have been proposed. See, e.g., U.S. Pat. No. 6,574,490; U.S. Pat. No. 6,865,408; U.S. Pat. No. 6,990,364; U.S. Pat. No. 7,133,710; each of which is incorporated herein by reference. The forearm can be a desirable site for tissue measurements for several reasons. As an example, mechanical systems can be devised that allow highly reproducible selection of sampling site, which can be important to help reduce measurement error arising from sampling of different tissue volumes. See, e.g., U.S. Pat. No. 7,206,623; U.S. Pat. No. 7,233,816; each of which is incorporated herein by reference. However, such mechanical systems can be unreliable if the locating hardware is displaced, and can be inconvenient for users, leading to low compliance and consequently low achieved sampling repeatability. Forearm (or other location) measurements without such tissue-mounted locating devices can allow the skin to be presented in conditions that vary across samples, for example by differing skin wrinkles or curvature, which can lead to tissue sampling variations that in turn can lead to decreased measurement accuracy. A forearm sampling site can also pose further difficulties, especially when comparing results to measurements obtained from blood from conventional “finger stick” meters. There is a need for tissue sampling methods and apparatuses that allow sampling of tissue, particularly skin, in ways that result in good optical measurement properties without the requirement for mechanical changes to or mounts on the skin. There is also a need for tissue sampling methods and apparatuses that allow consistent sampling of substantially the same tissue volume, particularly when sampling tissue of the hand.

### BRIEF DESCRIPTION OF THE DRAWINGS

FIG. 1 is a schematic illustration of an analyte measurement system according to the present invention.

FIG. 2 is a schematic illustration of an example embodiment of the present invention.

FIG. 3 is a schematic illustration of tissue features determined using an example embodiment of the present invention.

FIG. 4 is a schematic illustration of tissue features determined using an example embodiment of the present invention.

FIG. 5 is a schematic illustration of an example embodiment of the present invention.

FIG. 6 is a graphical representation of results obtained with an example embodiment of the present invention.

FIG. 7 is a schematic illustration of optical sampling at various sites.

FIG. 8 is a schematic illustration of an example embodiment of an apparatus according to the present invention.

FIG. 9 comprises graphs illustrating sampling performance obtained with example embodiments of the present invention.

### DESCRIPTION OF INVENTION

Optical sampling of tissue generally depends on interaction of light with various constituents of the tissue. For noninvasive analyte measurements, this involves the interaction of light with skin. Skin is a complex medium, with various layers and a wide variety of structures and materials present. An optical measurement of an analyte in skin thus must determine the interaction of light with the analyte, and be able to do so even in the presence of the many complexities of the skin itself. This can be done by taking optical measurements and corresponding reference measurements, and then determining a model that relates the optical measurement with the reference value. The model can then be used with future optical measurements to determine the analyte value without requiring a (usually) invasive reference measurement. This approach can accommodate complexities in skin structure, since those complexities that do not contribute to the analyte measurement can be addressed by the model. However, changes in the complexity of the skin can significantly complicate this approach, since a model determined for one skin structure can be inaccurate when applied to optical measurements taken from another skin structure. Also, given a region of skin, there can be portions of the skin that are better suited to optical measurement due to lesser contribution from complexities that interfere with optical measurement, e.g., thick calluses can reduce the amount of light that penetrates deep enough into the tissue to interact with an analyte such as glucose.

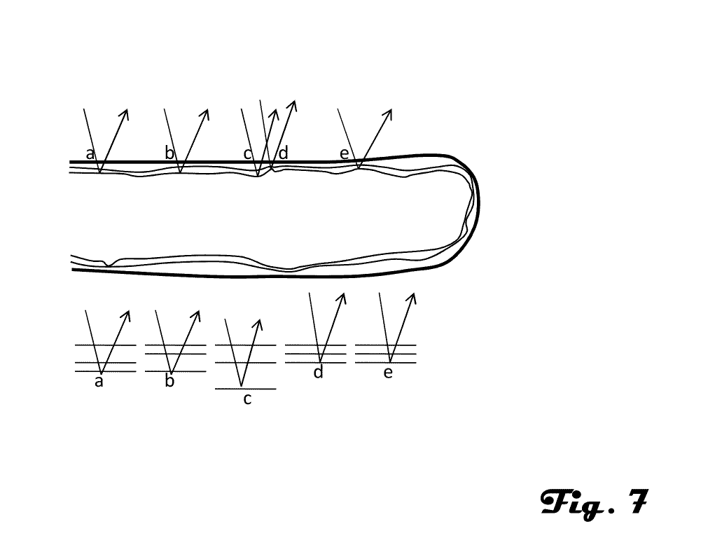

FIG. 7 is a simplified schematic illustration of a finger and the challenge of optically sampling at various sites. While the actual tissue structure of a finger is much more complex than that shown in the figure, for simplicity of illustration the figure assumes just two layers in the skin. The layers vary in thickness along the finger, for example due to calluses, wrinkles, scars, fat, etc. Representative (though simplified) optical paths as the tissue is sampled are shown at a, b, c, d, and e. Light at a passes through a thicker outer layer and then a relatively thinner inner layer. Light at b passes through a similar total thickness, but the outer layer at b is thinner relative to the inner layer. At c both layers are substantially thicker, perhaps due to a wrinkle or callus. At d and e both layers are thinner, perhaps due to a groove or scar or just a thin portion of the skin. A model that relates optical characteristics of the skin, applied to all 5 sampling sites, would have to be robust with respect to the large changes in tissue structure across the 5 sites. Also, assuming for the sake of illustration that analyte information is contained in the inner layer, then sampling site b would be preferred since the outer layer is thinner and the inner layer thicker. Also, since optical sampling is generally done over a region of skin and not just a point, regions with consistent skin composition such as around b would be desired over regions such as around c and d where the layer thickness varies greatly.

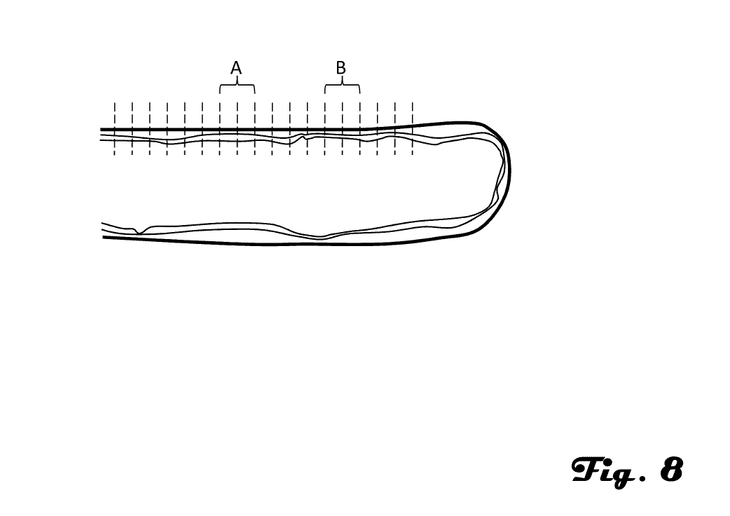

Embodiments of the present invention provide a method of optically sampling tissue such as the skin of the finger in a manner that encourages sampling from sites that have properties consistent with those used in determining a model, and from sites having properties that are consistent with good measurement performance. FIG. 8 is an illustration of such a method, using the simplified tissue illustration of FIG. 7. The tissue is optically sampled at each of a plurality of locations, shown as equally spaced along the length of the finger in the illustration although they can be along only a subset of the finger, and can be unequally spaced or spaced adaptively (e.g., closer together in regions that appear to be more promising as a sampling site). The number of locations and the distance between them can depend on the optical sampling system, e.g., upon the size of the region of tissue sampled by the system. Each sampling event does not need to be a full sampling (i.e., a sampling event long enough to generate an analyte measurement). Rather, each sampling event can be just long enough to gather sufficient optical information to determine metrics related to the desirability of the sampling site for the measurement. As an example, using the simplified illustration, two metrics can be determined: the strength of the signal related to the analyte, and the consistency of the optical signal over a region of the tissue. In the simplified finger, the signal strength metric is characterized by a thick inner layer and a thin outer layer; the consistency metric is characterized by lack of rapid change in the two thicknesses. Metrics over several sampling sites can be determined; the figure illustrates brackets over two regions each having three sampling sites. At regions A and B, the layers are not rapidly changing so the consistency metric will indicate A and B to be good sampling regions. The thicknesses of the inner and outer layers in region A will indicate a stronger signal metric, identifying region A (for example, the middle site in region A) as the desired sampling site.

In an example embodiment of the present invention, a hand sampling apparatus was used. A finger was positioned relative to the apparatus such that an initial sampling location near the distal knuckle of middle finger was presented to the optical sampling apparatus. The sampling site was rastered along the length of the finger such that 20 sampling sites were used. The sites were separated from each other by 0.75 mm. Each site was sampled for 6 seconds, and the optical information and sampling site location stored.

For determination of an appropriate sampling location a wide variety of metrics can be utilized. For purposes of illustration, spectral variance and a measure of the strength of the water signal at wavelengths that generally contain information relevant to glucose concentration were used. The metric for assessment of the water signal is referred to a Region 3 peak-to-trough and is calculated by comparing absorbance differences between the absorbance trough at about 4200 wavenumber versus the peak absorbance at around 5500 wavenumber. Larger values of this metric correspond to sampling regions where more relevant signal can be obtained. For illustration, a sliding window of three samples was used in the calculation of spectral variance and region 3 peak-to-trough. See, e.g., U.S. patent application Ser. No. 10/410,006, filed Apr. 9, 2003, “Reduction of errors in non-invasive tissue sampling” (incorporated herein by reference), for description of how to determine these and other metrics that can be suitable. Finding the window with the smallest spectral variance effectively locates the area of the finger where the spectral change is the smallest over the area of movement. This stability can minimize sampling error because all spectra within the area of the sampling site look very similar. Additionally, finding the window with the largest region 3 peak-to-trough locates the area of the finger where the peak-to-trough is largest over the area of movement, which is desired for the ideal sampling location.

Before calculating the mean spectral standard deviation across the wavenumbers for each window, the waterbands at 5200 and 6900 wavenumbers were removed. The average region 3 peak-to-trough of the 5200 waterband was also calculated according to standard equations. The preferred sampling location was selected based on the window with the minimum spectral variance and maximum region 3 peak-to-trough, according to the following rules:

a) If the two spectral metrics agree on the window, the middle location of this window is the preferred sampling site.
b) If the window with the minimum spectral variance is consecutive to the window with the maximum region 3 peak-to-trough, one of the overlapping points that is in both windows is the preferred sampling site (the middle location of the spectral variance window).
c) If the two spectral metrics do not agree on the window, the window with the minimum spectral variance takes precedence, and the middle location of this window is the preferred sampling site.

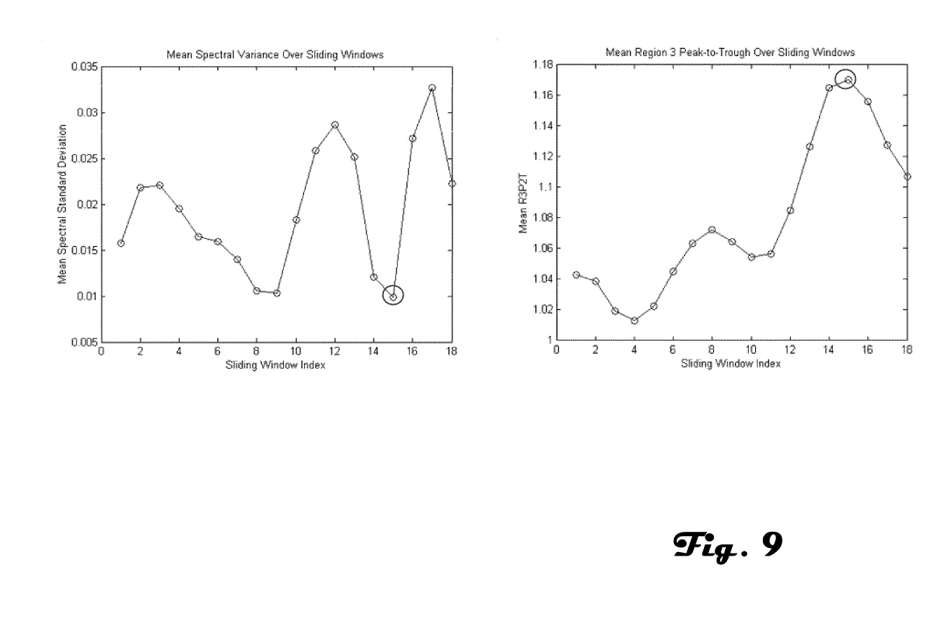

FIG. 9 comprises plots from an example subject that indicate the stability of a sampling site based on the minimization of spectral variance and maximization of region 3 peak-to-trough over a window of 3 consecutive sampling sites. The plot of mean spectral variance (on the left in the figure) corresponds to the stability of the tissue conditions—lower variance correlates with greater consistency between portions of the tissue as it interacts with light. From the figure, it can be seen that the tissue is consistent around sliding window indices 8, 9, 14, and 15. Those locations are accordingly locations where optical sampling would encounter consistent tissue conditions, and errors associated with unpredictable or too large variations in tissue conditions would be reduced.

The plot of mean region 3 peak-to-trough (on the right in the figure) corresponds to the strength of an optical signal at wavelengths that generally contain information relevant to glucose concentration—larger values of this metric correspond to sampling regions where more relevant signal can be obtained. From the figure, it can be seen that large values of this metric are found at sliding window indices 14, 15, and 16. The best combination of the two metrics occurs at sampling location 15. Once this location is determined, then a full optical measurement can be made at this location and the analyte concentration determined.

In some applications, finding a sampling location with a good match between a metric and a similar metric determined during model determination can be more important than finding the best sampling location. The same technique described above can be used, except that instead of maximizing certain metrics determined from the optical sampling data the match between those metrics and the desired values (e.g., from the calibration model determination) is optimized.

Embodiments of the present invention include apparatuses that sample a plurality of sites and determine which site is preferred according to metrics such as those described above. Such embodiments include optical sampling systems such as those described in U.S. Pat. No. 6,865,408 “System for non-invasive measurement of glucose in humans”, and U.S. Pat. No. 6,574,490 “System for non-invasive measurement of glucose in humans”, each of which is incorporated herein by reference. Such systems can be adapted to accommodate the present invention by inclusion of an ability to scan across a plurality of sampling sites, for example by steering the optics, by moving the optics, by moving the tissue automatically or by prompting the user, or a combination thereof. A control system can use results from the scanning of a plurality of sites to determine one or more preferred sites, and then use that site for subsequent sampling and analyte determination.

Embodiments of the present invention provide methods of determining tissue properties such as the concentration of analytes such as glucose in tissue. In such methods, a method like that described above is used to determine a preferred sampling site. Light is then directed to the preferred sampling site. Light expressed from the tissue in response to such incident light is then collected and analyzed to determine the property, such as the concentration of glucose in the tissue or a compartment thereof. Suitable methods of analysis include those described in patents assigned to InLight Solutions, Inc.

Embodiments of the present invention provide methods of building models relating incident light, expressed light, and analyte concentration. A preferred sampling site is determined using a method such as those described above. Light is then directed to the preferred sampling site on the skin, and light expressed from the tissue in response to such incident light is collected. A reference measurement of the analyte of interest is determined. The process is repeated a plurality of times, and the resulting combinations of incident light, expressed light, and analyte measurement used to produce a model relating the tissue's interaction with light with the analyte concentration, for example by using multivariate methods.

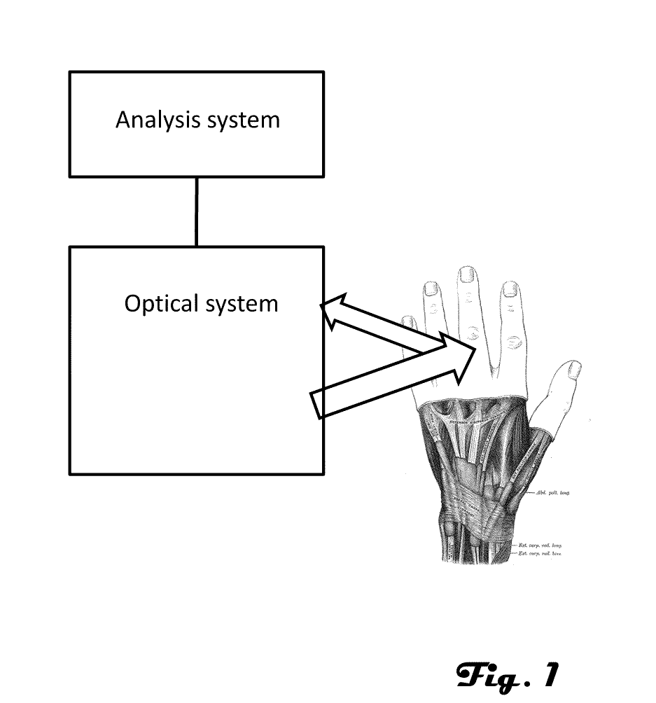

FIG. 1 is a schematic illustration of an analyte measurement system according to the present invention. An optical system communicates illumination electromagnetic energy to tissue to be sampled, and collects electromagnetic energy expressed from the tissue responsive to such illumination electromagnetic energy. An analysis system analyzes the collected light to determine properties of the tissue. In one example embodiment, the optical system comprises an illumination source and an illumination optical system adapted to transmit energy from the illumination source to the tissue, and a detection optical system to collect energy from the tissue and transmit it to a detector. The output of the detector is then analyzed by a programmed computer. Example optical systems that can be suitable for use with the present invention include those described in the patents and applications referenced elsewhere herein, and those described in patents and applications assigned to InLight Solutions and Sensys Medical. Some of those examples contemplate sampling on the forearm, and accordingly would be adapted to accommodate sampling on the hand as contemplated in the present invention. The present invention(s) include sampling on any body part that can be reliably positioned according to any of the methods and apparatuses described herein, although the descriptions that follow generally assume the hand or finger for simplicity of illustration. The present invention(s) allow substantially the same portion of the tissue to be sampled for measurement each time the tissue is presented to the system, even when the tissue is removed from the system and then reinserted hours or days later.

Optical measurement of glucose generally benefits from optical systems with high signal to noise. The tissue to be measured can be thought of as a part of a glucose measurement system, therefore any variation in the measured tissue contributes noise to the measurement. Because tissue is a heterogeneous medium, measuring at different sites on the tissue can introduce unwanted variation in the measurement results, therefore it is desirable to measure the same region of tissue for each person. The surface and outline characteristics of a person's hand and skin are very unique from person to person and can be used to precisely and accurately position (or reposition) a portion of tissue relative to an optical measurement system.

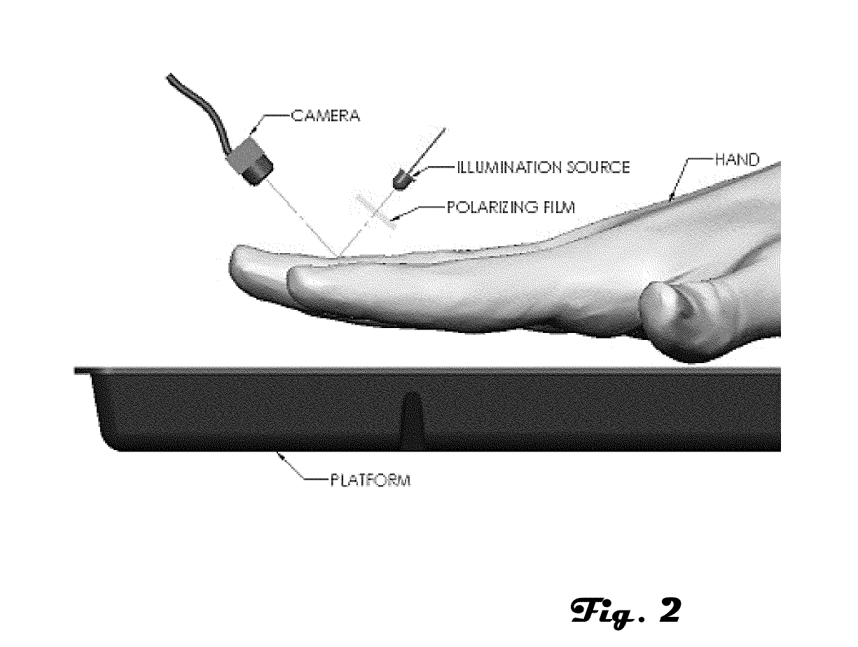

FIG. 2 is a schematic illustration of an example embodiment of the present invention. A hand sampling alignment system as shown in the figure interfaces with a measurement system (not shown) such as those described elsewhere herein. The hand sampling alignment system in the figure is shown as a separate optical system, although in some embodiments it can be integrated with the optical system used for measurement. In the example in the figure, an illumination source provides light that is directed through a polarizer (polarizing film in the figure) to the surface of the tissue (a hand in the figure). Light reflected from the tissue is then collected in an imaging manner by a camera. The image collected by the camera can then be used to adjust the relative positioning of the tissue and the measurement optical sampling system, for example by moving (translating, rotating, or a combination thereof) the support platform; by moving the optical measurement system; by moving alignment features such as those described in U.S. provisional application 61/111,815, filed Nov. 6, 2008, incorporated herein by reference; and by communicating to the user to encourage user-initiated alignment (e.g., instructions such as “move hand to the left” or a visual alignment cue such as a crosshairs target or overlaid images whose alignment corresponds to the desired positioning).

As a specific example of operation with a system like that in FIG. 2, tissue is illuminated with the light source and an image of the tissue is recorded using a camera and the image is digitized and read into a computer system for subsequent processing. The illumination system can be designed to enhance the contrast of surface features on the skin. Surface features can be large-scale, like skin edges or knuckle ridges or finger nail boundaries, or small scale, like skin wrinkles or hair follicles or other surface morphologies, or subsurface, like vein or capillary positions, or combinations thereof. Also, marks can be made on the skin and used in place of or in addition to surface features.

A computer system can execute an algorithm to extract the surface features from the noisy background of the unprocessed image. The algorithm can comprise simple edge detection techniques, like Matlab's edge function, or more advanced methods designed for the particular characteristics of the desired tissue location or optical sampling system. The output of the algorithm can be displayed to a user and the user repositions their hand based on the displayed information. This process can be repeated until the hand is positioned within some predetermined tolerance to the desired location and the user is instructed to remain motionless. Alternatively, the output of the algorithm can be passed into another algorithm that calculates the error in hand position, converts this error into a set of displacement factors, then controls actuators to move a platform on which the hand is resting or the optical measurement system or components thereof to achieve the necessary alignment of the tissue. As examples, a metric used to quantify the positioning error can be simple absolute overlap of the location of features relative to a target position, or determined as a percentage area of overlap between the actual tissue position and the target tissue position normalized by the area of tissue sampled by the optical sampling system. The process can be repeated multiple times to improve the positioning accuracy, and can in some embodiments be a continuous control process by continuing the control of the relative position while the analyte measurement is being done.

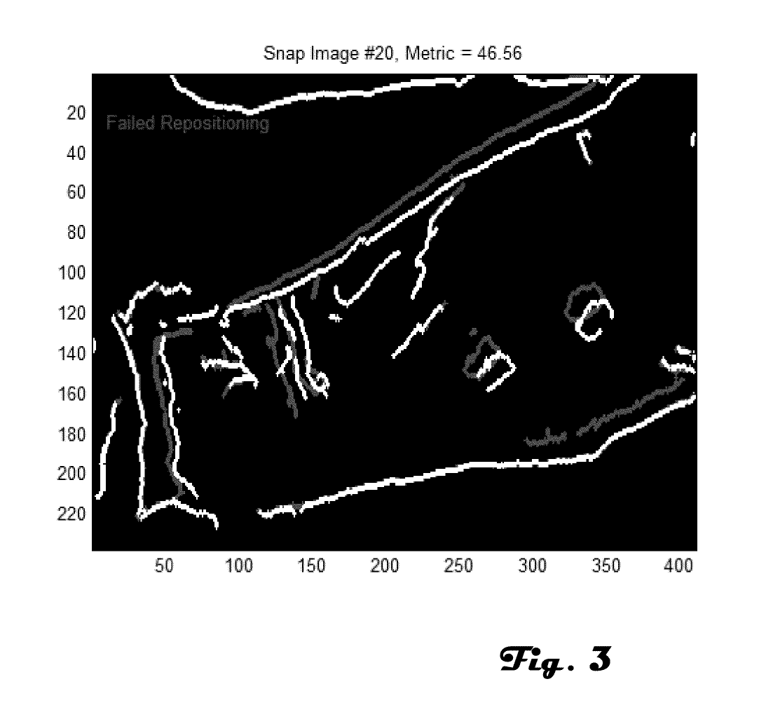

FIG. 3 is an illustration of a non-aligned sampling presentation (where a “presentation” is an event at which the tissue to be sampled is presented to the alignment system). Edges, ridges, marks, or other distinguishing features of a finger are shown in white for a base sampling presentation. Similar detected features at a different sampling event are shown in red. The two sets of detected features demonstrate that the tissue is not presented at the same location (position, orientation, or both) in this sampling presentation as the base sampling presentation. Alternatively, the misalignment can be the result of presenting different tissue (e.g., a different finger, or a finger of a different person). In either case, the misalignment indicates that the optical sampling system would not be sampling the same tissue volume as in the base sampling presentation, and accordingly would produce an analyte measurement with undesirable error. The images in FIG. 3 can be displayed directly to the user to allow the user to reposition the tissue until desired alignment of the detected features was achieved. Alternatively, the information in FIG. 3 can be used to present instructions to the user such as “move finger up” or to present an integrated graphical image such as symbol (e.g., a crosshairs or a geometric shape) whose alignment relative to a graphical target shape indicates required motion of the hand to achieve the desired alignment. Alternatively, the information in FIG. 3 can be used as input to an alignment control system (e.g., a computer-implemented control system) to determine and control motion of the optical sampling system or portions thereof or motion of support or alignment features in contact with the hand, or selection of portions of a large area sampled by the optical sampling system.

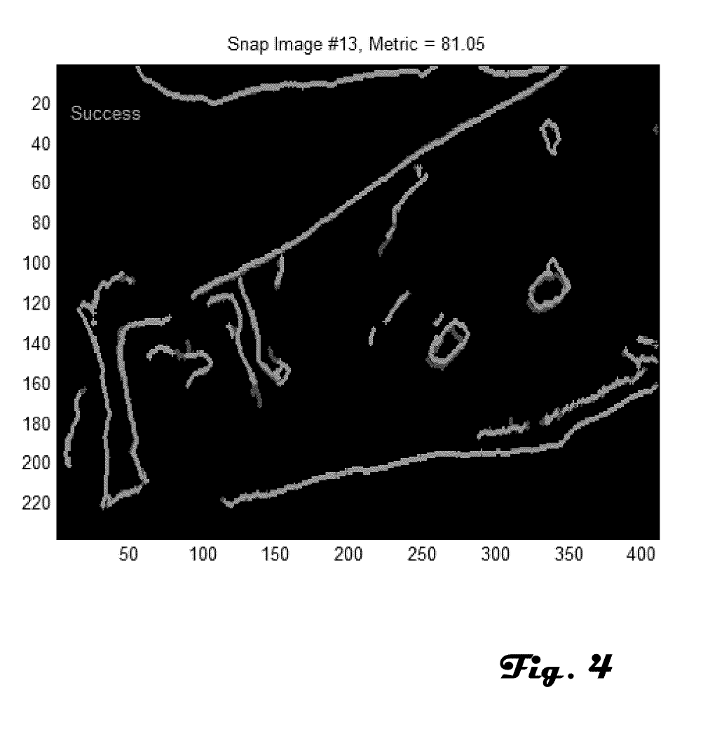

FIG. 4 is an illustration of a sampling presentation aligned with a base sampling presentation. As with FIG. 3, edges, ridges, marks, or other features have been detected for a base sampling presentation and for the current sampling presentation. Where the features are acceptably aligned, they are depicted as green in the image. Where they are not acceptably aligned, the image shows a red line. In aggregate, the alignment of the features in the two sampling presentations is acceptable (e.g., within some error metric such as percent aligned, or integration of total distance between the detected features, or a weighted determination where some features have greater importance than others, or other difference or distance metrics known to those skilled in the art). The optical sampling system will accordingly be sampling substantially the same tissue volume with the current sampling presentation as in the base sampling presentation, and errors due to tissue misalignment will be within an acceptable range.

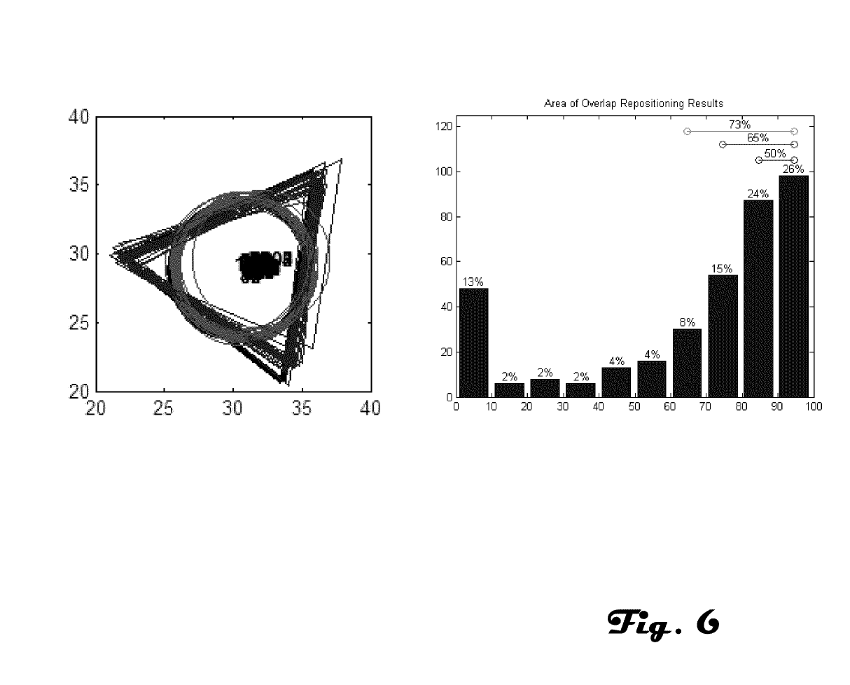

FIG. 6 is an illustration of the operation of an example embodiment of the present invention using three marks disposed to form a triangle on the skin. The red circle is the area sampled by the optical sampling system; the blue triangles are positions of unique skin features for repeated positioning of the tissue. In a test of the embodiment, the triangle formed by the marks was used to facilitate alignment of the tissue by either moving the optical sampling system or by instructing the user to move the tissue. As can be seen by the overlap of the triangles and the circles, the example embodiment was successful in encouraging the sampling of substantially the same tissue portion on multiple sampling presentations. The “Area of Overlap Reposition Results” graph shows typical results from one embodiment of the invention for a number of people and tissue presentations. As can be seen from the graph, the large majority of sampling presentations resulted in very high degree of overlap of the tissue are sampled.

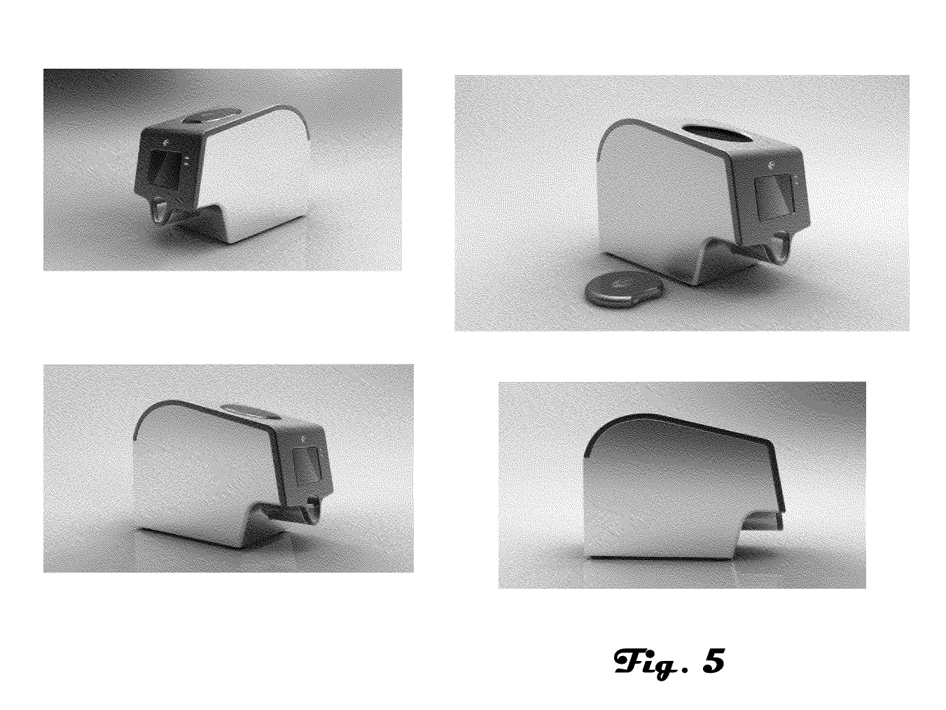

FIG. 5 is a schematic illustration of an example embodiment of the present invention. An optical sampling system is contained within the housing shown in the figure. A display capable of communicating instructions and results to a user is located on the front of the housing. A finger of the hand is presented to the optical sampling system by inserting the finger in the opening below the display on the front of the housing. A tissue imaging system such as that described above is mounted with the housing such that the surface of a finger inserted in the opening is presented to the imaging system. The position of the optical sampling system within the housing relative to the finger can be controlled by a control system to minimize the alignment error, as described above. Alternatively, the shape and position of the channel defining the opening can be controlled to change the position of the finger such that alignment error is minimized as described above. Alternatively, the display screen can be used to communicate to the user information that allows the user to move the finger to minimize alignment error.

The present invention(s) also provides methods of determining tissue properties, such as the presence or concentration of analytes, including measurements of glucose concentration in tissue. An example embodiment of such a method comprises supplying an alignment apparatus such as those described or enabled herein, in operative relationship to an optical sampling system. A user presents a hand to the alignment system which fosters a desirable, repeatable positioning of the tissue to the optical sampling system, and the optical system provides illumination energy and detects light expressed from the tissue responsive to the illumination energy. Illumination energy and detected light can comprise visible light, heat, infrared light, ultraviolet light, mid-infrared light, near-infrared light, other forms of energy or wavelengths of light, and any combination or subset thereof. An analysis system analyzes the collected light and determines the tissue property, e.g., the glucose concentration, for example using multivariate methods such as those described in the patents and applications incorporated herein. The user subsequently presents the hand to the alignment system, and the alignment system facilitates positioning of the hand such that substantially the same tissue portion is presented to the optical sampling system as in a base or other previous sampling presentations, and the illumination, detection, and analysis steps are repeated.

The present invention also comprises methods for making tissue measurement systems, such as noninvasive glucose measurement systems, comprising making an alignment system according to the present invention, making an optical system suitable for use with the alignment system and suitable for determining interaction of the tissue with light sufficient to determine the tissue property, making an analysis system such as a multivariate analysis system, and integrating the aforementioned elements into a tissue measurement system.

The present invention(s) can also provide sensing capability combined with the above-described alignment capability. For example, following insertion of the body part into the optical system, the system can detect if the body part is aligned correctly. If the body part is in an acceptable position the system initiates an optical scanning or measurement process. The scanning or measurement process continues until either the measurement has been completed or the system detects movement or misalignment of the body part. If misalignment is detected, then the system can notify the user and can provide prompting messages to re-align the body part. Upon realignment the system can automatically start the measurement process. In use the measurement process can be re-started or simply continued (retaining measurements made during times of acceptable alignment) until enough measurements have been made.

The present invention has been described by way of various example embodiments. It will be understood that the above description is merely illustrative of the applications of the principles of the present invention, the scope of which is to be determined by the claims viewed in light of the specification. Other variants and modifications of the invention will be apparent to those of skill in the art.

We claim:

1. A method of selecting a preferred sampling location for optical sampling of tissue, comprising determining a plurality of spectral metrics for each of a plurality of sampling locations, and selecting one of the plurality of sampling locations as the preferred sampling location responsive to the values of the plurality of metrics each of the plurality of sampling locations.

2. A method as in claim 1, wherein one of the plurality of spectral metrics comprises spectral variance.

3. A method as in claim 1, wherein one of the plurality of spectral metrics comprises peak-to-trough.

4. An apparatus for determining a preferred sampling location for optical sampling of tissue, comprising an optical sampling system, and an analysis system operating according to the method of claim 1.

5. A method of determining a tissue property, comprising selecting a preferred sampling location according to the method of claim 1, then determining the tissue property from the response of tissue at the preferred sampling location to incident light.

6. A method as in claim 5, wherein the tissue property comprises the presence or concentration of an analyte in the tissue.

7. A method as in claim 6, wherein the analyte comprises glucose.

8. A method of precisely aligning a person's hand relative to an optical sampling system by optically measuring characteristics of the hand or a portion of the hand.

9. A method as in claim 8 where the hand is precisely aligned to a previously recorded position by optically measuring surface characteristics of the skin on the hand that is unique to that person.

10. A method as in claim 8 where the hand is precisely aligned to a previously recorded position by optically measuring the outline of the hand or a portion of the hand.

11. A method as in claim 8 where the user is provided visual information that enables the user to accurately position the hand relative to an optical measurement system.

12. A method as in claim 8 where a polarizing film is employed in the optical measurement system to suppress surface reflections from a previously applied smoothing agent.

13. A method as in claim 8, further comprising comparing the determined characteristics with similar characteristics determined at a different presentation of the tissue to the alignment system.

14. A method as in claim 8, wherein the characteristics comprise one or more of: edges of the tissue, edges of a hand or finger, boundaries of a finger nail, ridges in the skin, grooves or wrinkles in the skin, marks placed on the skin, tattoos on the skin, hair follicles, subcutaneous features, vein or capillary positions.
